(19) United States
(12) Patent Application Publication
(10) Pub. No.: US 2004/0169819 A1
(43) Pub. Date: Sep. 2, 2004

(54) Human eye glare tester

(76) Inventor: Takuro Tsukatani, Scottsdale, AZ (US)

Correspondence Address: Ellis & Venable P.C., Suite 1875, 101 North First Ave.,, Phoenix, AZ, 85003, US

(21) Appl. No.: 10/753,946

(22) Filed: Jan. 7, 2004

Related U.S. Application Data
(60) Provisional application No. 60/438,392, filed on Jan. 7, 2003.

(51) Int. Cl. A61B 3/10

(52) U.S. Cl. 351/213

(57) ABSTRACT

An apparatus and method for testing a human eye's sensitivity to glare under various conditions where light intensity is varied and passes through a given translucent filter to create a glared condition. The human eye is then provided a filter such as a pair of sunglasses that with the proper level of filter may assist a human subject in eliminating the glared condition.

This application claims benefit of provision application serial No. 60/438,392.

### FIELD OF THE INVENTION

The present invention relates generally to eye testing and, in particular, to an inexpensive glare tester that simulates a wide range of glare conditions caused by such sources as sunlight, fluorescent light, and automotive headlights.

### BACKGROUND OF THE INVENTION

As people get older, their eyes can become more sensitive to glare. When this sensitivity becomes more acute, many individuals begin to have difficulties from the glare caused by sunlight, as well as other light sources such as fluorescent lights and automotive headlights. Accordingly, it is important to know how an individual's vision is affected by glare, so that appropriate measures may be taken to correct or compensate for the increased sensitivity to glare.

Existing measurement devices, including those used to test for acuity, contrast sensitivity, and the like, have failed to adequately simulate glare conditions. As a result, it has not been easy for ophthalmologists or optometrists to take susceptibility to glare into account. This can be very problematic in that vision problems or serious eye disease, such as cataracts, glaucoma, diabetic retinopathy, retinitis pigmentosa, macular degeneration, and the other maladies may arise without accurate screening.

Broadly, this invention fills a need in the existing art, by providing an inexpensive device that may be used to simulate glare conditions. In terms of preferable features, the different embodiments of the invention include the ability to change the level of glare by altering the strength of a light source and/or changing the characteristics of a filter or other optical element or by changing the way in which light is diffused, reflected, or otherwise redirected or modified.

### SUMMARY OF THE INVENTION

Broadly, it is an object of the present invention to provide an inexpensive testing apparatus that measures an individual's sensitivity to glare.

It is a further object of the present invention to provide a method of testing an individual's sensitivity to glare.

It is a further object of the present invention to utilize a translucent image as the focal point where the glare is created.

It is a further object of the present invention to utilize a number of varying images in order to test different conditions under which glare may be present.

It is a further object of the present invention to utilize a varying light source that can penetrate the translucent image thus creating a glared condition.

The description of the invention which follows, together with the accompanying drawings should not be construed as limiting the invention to the example shown and described, because those skilled in the art to which this invention appertains will be able to devise other forms thereof within the ambit of the appended claims.

### BRIEF DESCRIPTION OF THE DRAWINGS

FIG. 1 is a frontal view of the apparatus;

FIG. 2A is a front view of a translucent filter;

FIG. 2B is a side view in cross section;

FIG. 3A is a frontal view of the filter with an image set with a lighting fixture;

FIG. 3B is a top view of the apparatus with the image set in the lighting fixture the holder and the image on the transparent or translucent sheet;

FIGS. 4A, 4B, and 4C are front views of examples of illustrations on translucent testing filters; 4A for acuity, 4B for contrast and 4C for night driving;

FIG. 5A is a frontal view of an alternate embodiment of the translucent filter wherein as shown in FIG. 5B the attachment hole such that one can attach multiple filters on the lighting fixture and interchange as it is needed; and

FIGS. 6A and 6B shows the front and side view of the lighting fixture where its angle can be adjusted so that the light goes directly toward a patient's eyes.

### DESCRIPTION OF AN EMBODIMENT

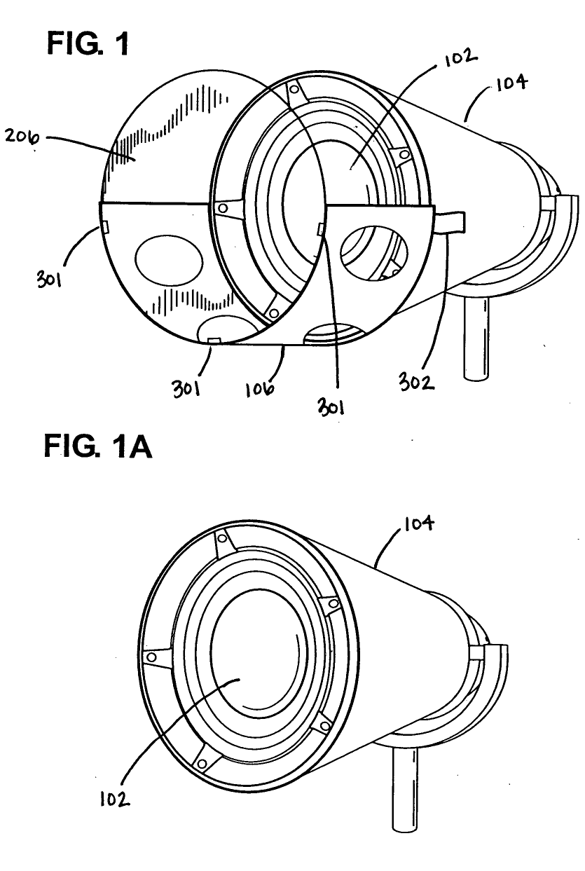

By way of one example of many to serve as background in understanding the present invention, FIGS. 1A and 1B show a replaceable translucent filter 108 that is attached at the top of the filter to a holder 106 which is part of a housing 104. The holder 106 is configured in such a way that the translucent filter 108 may be interchangeable and secured within the holder 106. The housing 104 in the preferred embodiment is a trapezoidal-shaped cone, which includes a light source 102. The light source 102 is a source, which may vary in luminescence by means of a potentiometer 110. The light source 102 must be directed toward the front portion of the housing 104 such that when charged above a minimal level of luminescence, the light emitted from the light source 102 will shine through the translucent filter 108 thus creating a condition of glare to a person viewing the filter 108 from the front of the filter 108.

The filters 108 generally comprise various testing charts or illustrations on a transparent sheet. They may also include color or optical characteristics to simulate different glare conditions. The light emitted from the light source 102 passes through the filter 108 that creates an image under glare. The filters 108 can be combined to produce different lighting situations.

A patient views the filter 108 through which the light is coming toward the patient's eyes. The patient can be tested using various intensities of sunglasses or color lenses to determine which color or density are most suitable for the patient. Additionally, eye doctors can utilize the device to measure a patient's vision changes under varying degrees of glare, which may lead to an early detection of eye diseases.

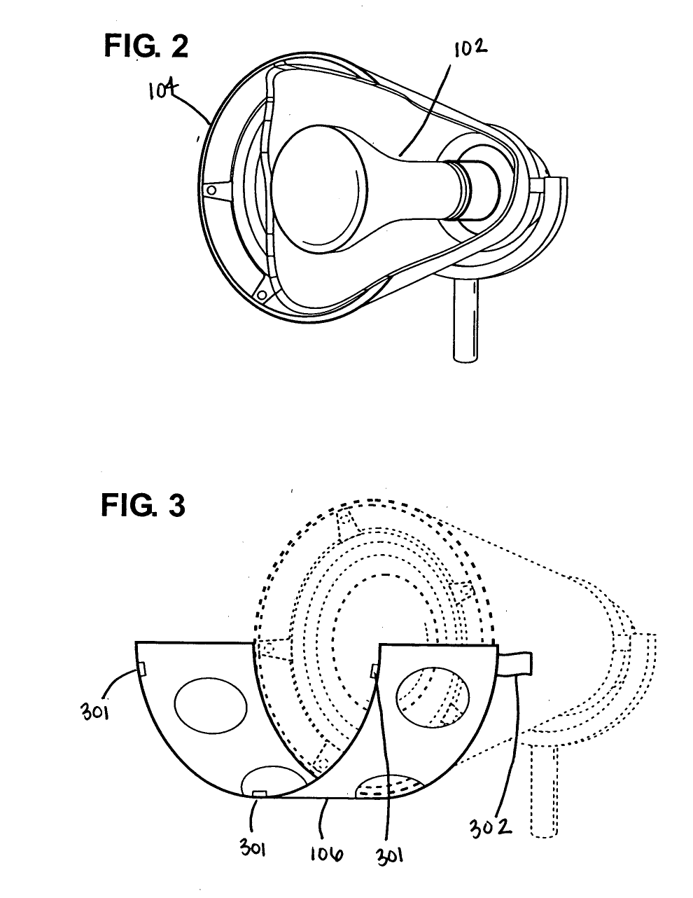

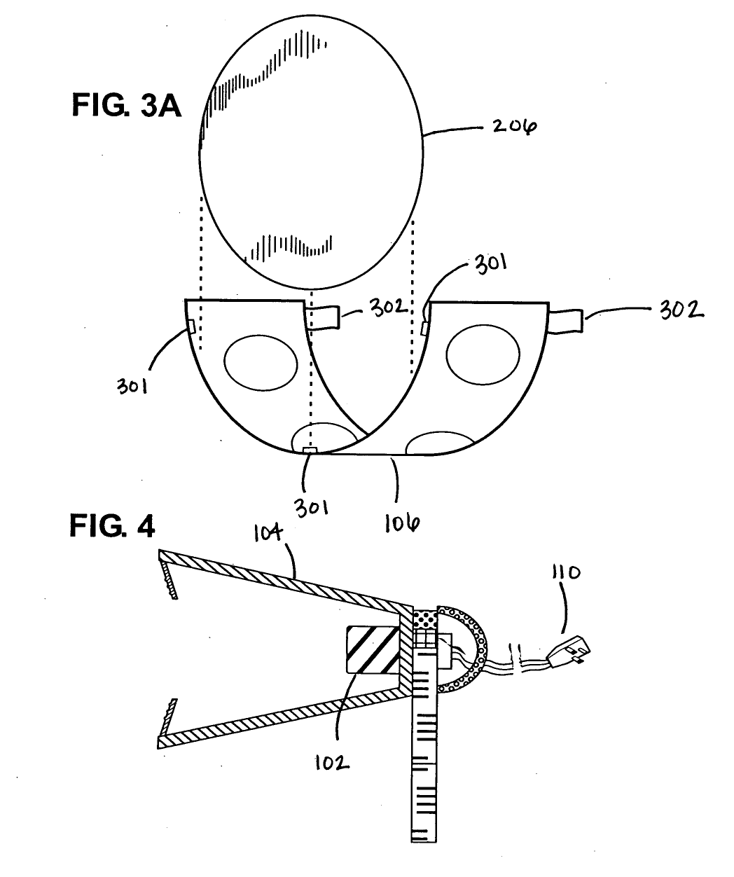

As shown in FIG. 2A, the front view of the filter 108 includes a hole 202 that attaches to the housing 104, and a securing means 204 that secures the translucent sheet 206. FIG. 2B shows the side view of the same elements. FIGS. 3A and 3B show the identical elements of the hole 302 that attaches to the housing 104 and a securing means 304 that secures to a translucent sheet 306 with the translucent sheet 306 including the additional elements of a scenic view embedded within the translucent sheet 306 showing the light source 102 within the housing 104. The light source 102 shines the beam of light through the translucent sheet 306 and creates a certain amount of glare which is seen by the human eye 308.

As shown in FIGS. 4A, 4B and 4C, each of the translucent sheets 406 may include distinct patterns or images that measure varying aspects of an individual's sensitivity to glare for acuity, contrast and night driving respectively.

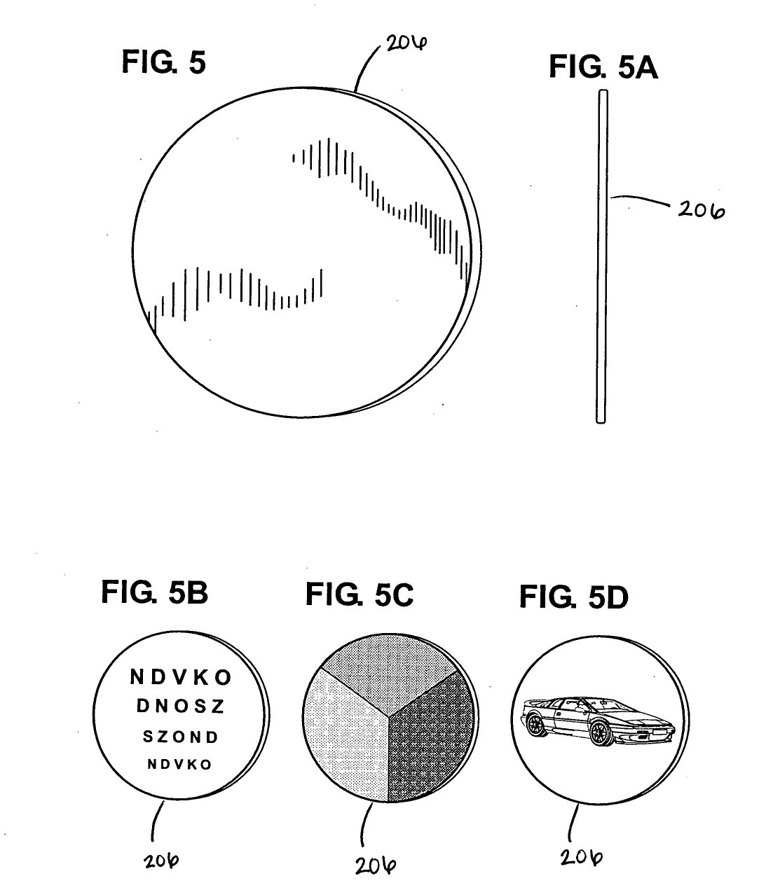

FIG. 5A shows an alternate embodiment of the shape of the attachment hole 502 such that the holder can include flexible material 504 so that one can attach multiple filters on the lighting fixture and interchange it as needed as shown in FIG. 5B. This is done much in the same way that and optometrist uses a series of interchangeable lenses that pivot along a single axis that allows the doctor to measure near or farsightedness. This would make the testing easier since it is not necessary to replace the filters each time.

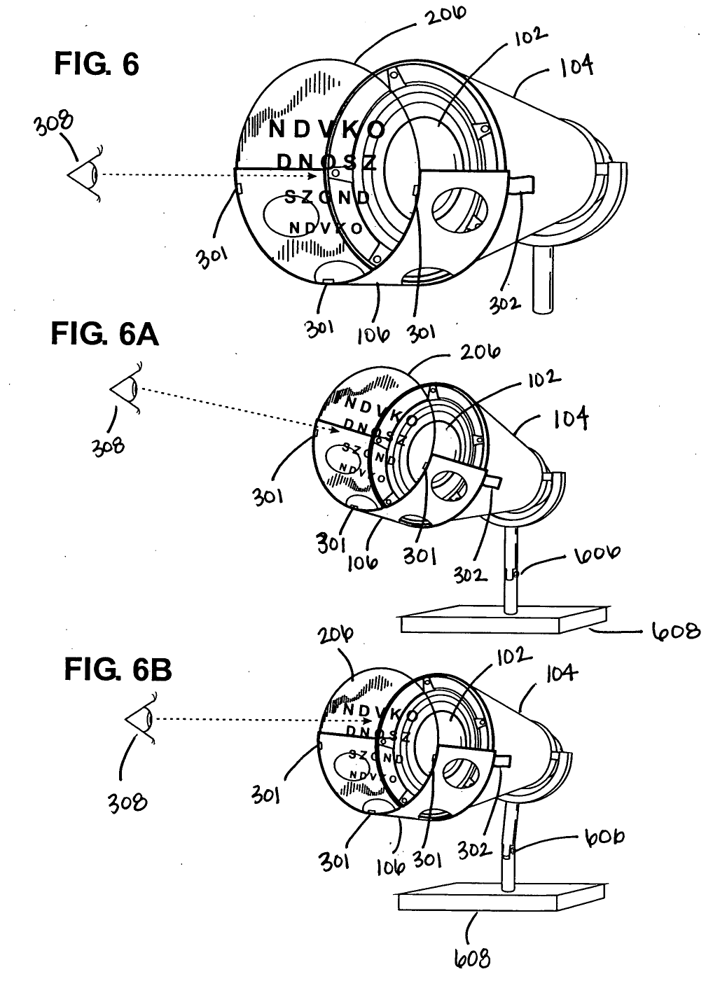

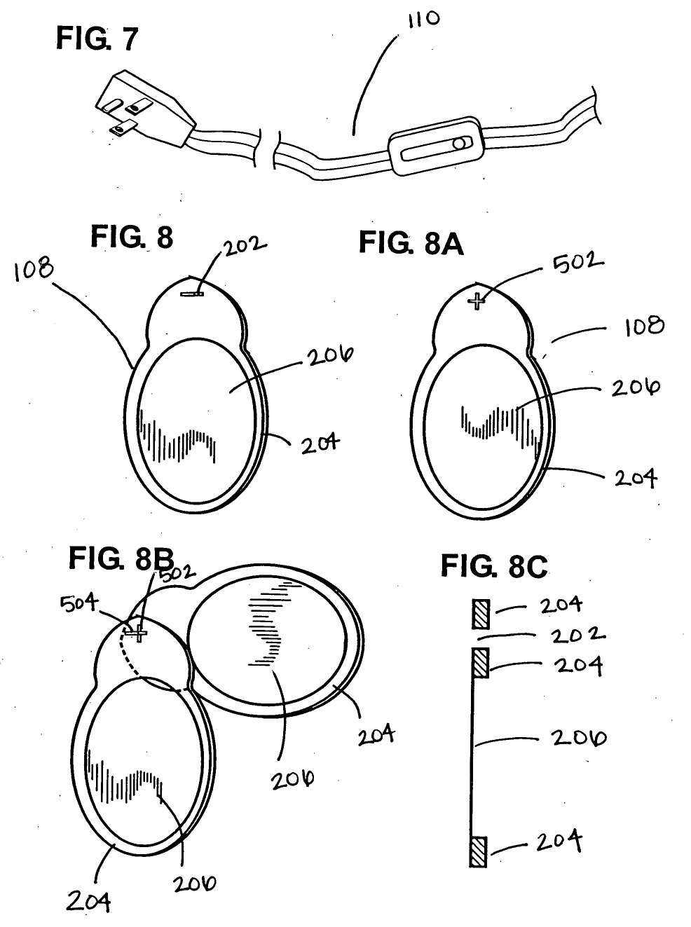

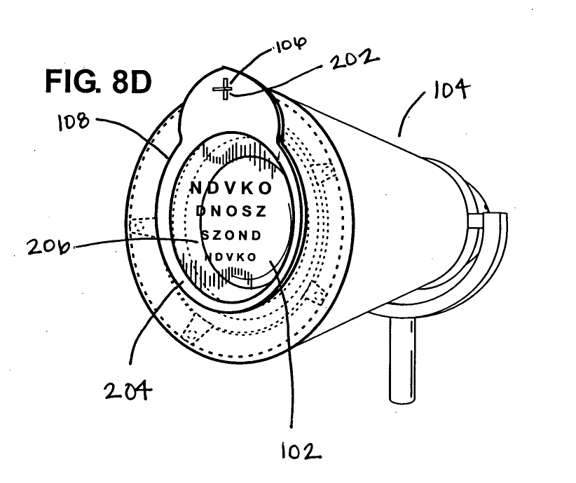

FIGS. 6A and 6B show the front and side view of the housing 604 resting upon a stand 608 where its angle can be adjusted along a pivot point 606 so that the light source 602 can be focused directly toward a patient's eye 308.

While the apparatus for practicing the within inventive apparatus, as well as a method of testing herein shown and disclosed in detail is fully capable of attaining the objects and providing the advantages hereinbefore stated, it is to be understood that it is merely illustrative of the presently preferred embodiment of the invention and that no limitations are intended to the detail of construction or design herein shown other than as defined in the appended claims.

Although the invention has been described in detail with reference to one or more particular preferred embodiments, persons possessing ordinary skill in the art to which this invention pertains will appreciate that various modifications and enhancements may be made without departing from the spirit and scope of the claims that follow.

What is claimed is:

1. An apparatus for measuring the human eye's sensitivity to glare comprising:
a. A housing, said housing further comprising an open end and a closed end;
b. A holder;
c. At least one translucent filter;
d. A source of light, said source of light coupled to the closed end within said housing such that said light source emits light in the direction of the open end of said housing;
e. Said translucent filter being secured within said holder such that when said holder is coupled to said open end of said housing, the translucent filter is positioned so that the light emitted from said light source passes in the direction normal to the translucent filter thereby creating a glared condition on the translucent filter.

2. The apparatus of claim 1 wherein said light source further comprises a varying amount of luminescence

3. The apparatus of claim 1 wherein said source of light further comprises a control to vary the amount of luminescence such that the light source emits.

4. The apparatus of claim 1 wherein said translucent filter further comprises at least one indicia inscribed on said filter.

5. The apparatus of claim 1 wherein said translucent filter is substantially circular in shape.

6. The apparatus of claim 1 wherein said at least one translucent filter is coupled at a pivot point such that said emitted light from said light source may pass through one or more at least one said translucent filters.

7. The apparatus of claim 1 wherein said housing is coupled to a stand such that said stand may rotate along an axis thereby changing the angle of the light emitted from said light source relative to a stationary human eye.

8. A method of testing a human eye's sensitivity to glare comprising the steps of:
a. Shaping a piece of translucent material;
b. Securing a light source in a housing having an open end and a closed end;
c. Positioning said translucent material in front of said light source at the open end of said housing;
d. Turning on said light source such that light is emitted from said light source and passes through said translucent material such that a glared condition is created at said translucent material;
e. positioning a semi-translucent filter between the translucent filter and a human eye that eliminates the glared condition as it appears to said human eye.

9. The method as described in claim 8 wherein said light source has a variable intensity of luminescence.

10. The method as described in claim 8 further comprising the step of securing a control to said light source such that said control may vary the intensity of the luminescence from said light source.

11. The method as described in claim 8 further comprising the step of varying the intensity of the luminescence from said light source

12. The method of claim 8 wherein said translucent filter is made of a plastic material.

13. The method of claim 8 wherein said translucent filter is made of a glass material.

14. The method of claim 8 further comprising the step of inscribing an indicia on the translucent filter.

15. The method of claim 8 further comprising the step of positioning a second translucent filter to said translucent filter thereby enabling the light emitted from said light source to pass through both the translucent filter and the second translucent filter.
